(19) United States
(12) Patent Application Publication
(10) Pub. No.: US 2011/0052675 A1
(43) Pub. Date: Mar. 3, 2011

(54) Orally Disintegrting Forms for Sustained Release and Controlled Release

(76) Inventor: Eric Yueh-Lang Sheu, Lafayette, CA (US)

(21) Appl. No.: 12/870,350

(22) Filed: Aug. 27, 2010

Related U.S. Application Data
(60) Provisional application No. 61/238,147, filed on Aug. 29, 2009.
(60) Provisional application No. 61/318,370, filed on Mar. 28, 2010.

(51) Int. Cl. A61K 9/127; A61K 9/14; A61K 9/22; A61K 8/06; A61K 9/16

(52) U.S. Cl. 424/450; 424/489; 424/465; 977/735; 977/906

(57) ABSTRACT

Orally disintegrating forms with sustained release capability and a method of making thereof are provided.

### CROSS-REFERENCE TO RELATED APPLICATION

This application claims priority to U.S. Provisional Application No. 61/238,147 filed Aug. 29, 2009 and U.S. Provisional Application No. 61/318,370 filed Mar. 28, 2010, both of which are incorporated by reference herein.

### FIELD OF THE INVENTION

This invention relates to orally disintegrating dosage forms with sustained release and controlled release capability and a method of making thereof.

### SUMMARY OF THE INVENTION

The invention relates to orally disintegrating tablets with sustained release capability and a method of making thereof. Microscopic sustained release (MSR) or microscopic controlled release (MCR) units of about 500 microns or less are made. This is achieved by incorporating drug(s) and release rate controlling excipients into pre-constructed pores of an excipient or a combination of porous excipients. These MSR or MCR units are then linked by proper binders and formed into suitable forms such as tablets, pellets, or granules. Upon administration to oral cavity, the tablet rapidly dissolves, leaving the MSR or MCR units moving down the enteric route to release the drug in a controlled fashion.

Sustained release and controlled release are provided in many dosage forms, for example, tablets, capsules, beads, gels, semi-solids, microparticles, nanoparticle, and liposome. Orally disintegrating forms such as orally disintegrating tablets (ODTs) in a sustained release or controlled release form do not require water to assist in oral administration, which is an essential benefit for those who have difficulty swallowing pills. However, most ODT products release immediately their drug contents following a rapid dissolution in the oral cavity and lead to fast pharmacokinetic effects. When a drug has short half-life and a sustained release pharmacokinetic effect is needed, ODTs with sustained release capability would benefit those patients who have problems swallowing. Also, a controlled release dosage forms are needed for drugs that may irritate stomach. Some diseases that occur in elderly population often need drugs in the ODT dosage form to improve patient compliance. If a sustained release or controlled release property is also required for those diseases, an ODT sustained release or controlled release dosage form becomes essential.

A drug and an excipient or a combination of excipients is provided that can sustain the release of the drug into pre-constructed molecular cages or pores of size greater than about 5 nanometers and less than about 500 micro meters to form a micro sustained release unit (MSR). These units are then pressed together with excipients to form orally disintegrating forms such such as tablets, pellets, or granules. Upon disintegration in the oral cavity, these MSR units follow the enteric route and release the drug in a sustained fashion. Similar process can be used for making orally disintegrating controlled release dosage form by pre-constructing a micro controlled release (MCR) unit and followed by formulating with excipients with orally disintegrating characteristics.

### DETAILED DESCRIPTION OF THE DRAWINGS

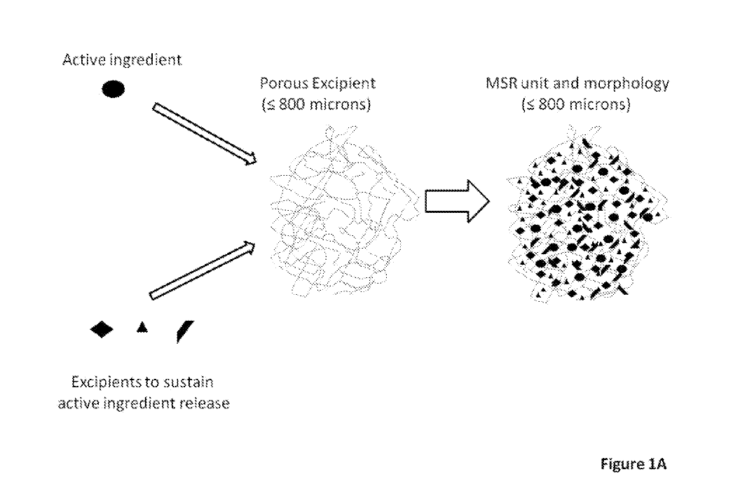

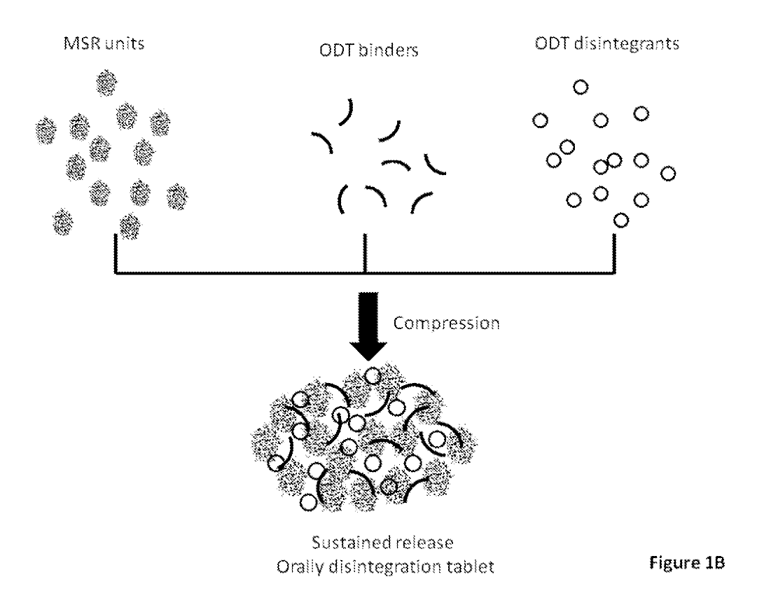

FIGS. 1A and 1B shows an embodiment of the present invention.

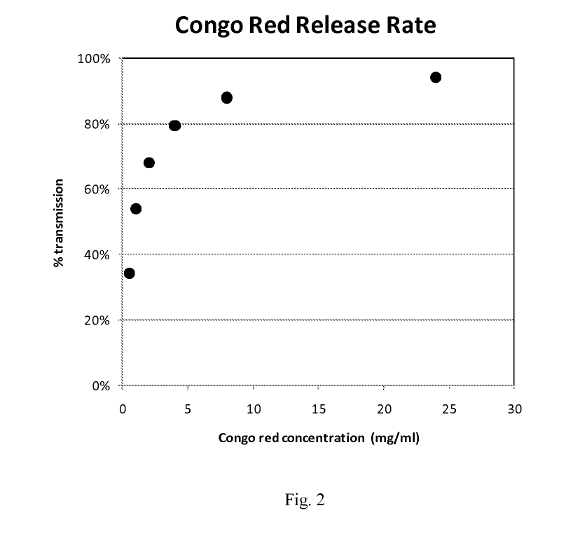

FIG. 2 shows an exemplary result of an embodiment of the present invention.

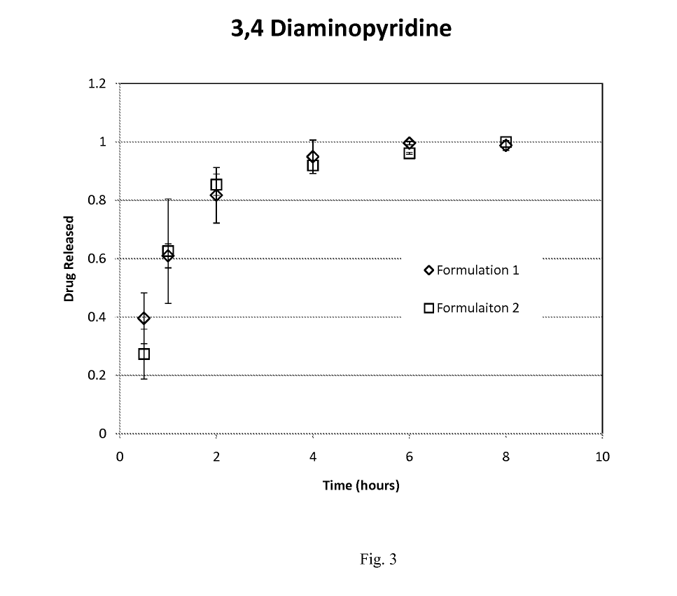

FIG. 3 shows a graph of Drug Released versus time of 3,4, Diaminopyridine of two different formulations.

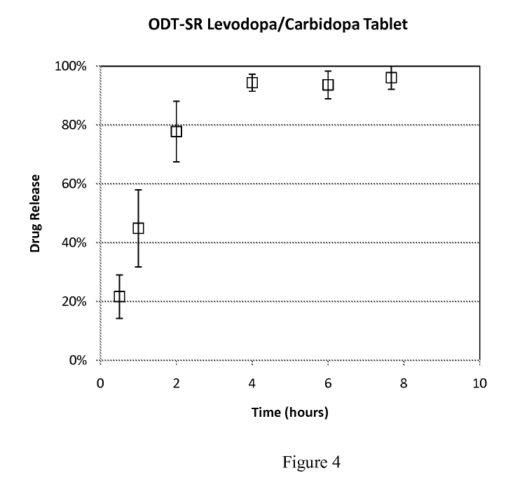

FIG. 4 shows a graph of drug releases versus time of an ODT-SR Levodopa/Carbidopa tablet.

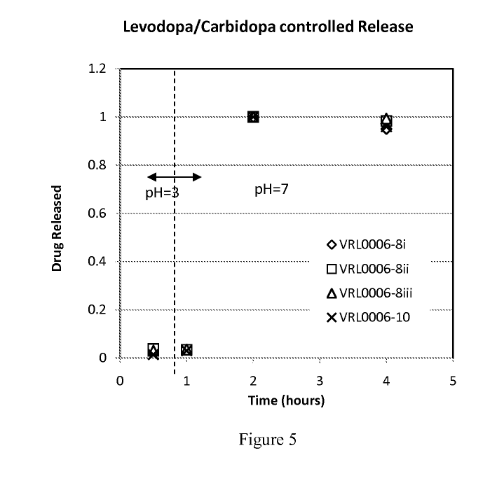

FIG. 5 shows a graph of controlled release of Levodopa/Carbidopa versus time.

### DETAILED DESCRIPTION OF THE INVENTION

Sustained release orally disintegrating dosage forms such as orally disintegrating tablets (ODT) and a method of making thereof are provided. The concept behind the ODT tablets and the method is to divide the sustained release ODT into two morphologically different parts. The first part is the sustained release or controlled release part including many micron size entities with sustained release or controlled release capability, referred to as microscopic sustained release (MSR) or controlled release (MCR) units. The morphology depends on how these MSR or MCR units are made. The second part is made by compressing these MRS or MCR units together with ODT binders, fillers, disintegrants, and other necessary excipients into tablets, which, upon exposure to water rapidly disintegrate into MSR units.

To make the MRS or MCR units, the active ingredients and excipients for sustained release are incorporated into pre-formed porous structures of micron size sustained release or controlled release units. The processes to incorporate active ingredients and excipients into the pre-formed porous micron structure can be through heating, cooling, pressurization, and/or solvent impregnation. These processes produce micron size sustained release or controlled release matrix units within the pre-formed porous structure. The process and the morphology of MSR units are shown in FIG. 1A.

In one embodiment, these units can be made by dissolving both drugs and excipients, where the MSR units control the drug release, into the pores or molecular pores of an excipient or a combination of several excipients. The size of the MSR units depends on the particle size distribution of the porous excipients ranging from about 5 nanometers to about 500 microns. The porous excipient can be about 5 to about 300 microns to avoid granular sensation when the tablet dissolves in the oral cavity. The porous excipients include, but not limited to, silica, polymers, mannitols, sorbitols, xylitols, fructose, lactose, starch, celluloses, guar gum, gelatins, Bucky balls, liposome, crystalline, polycrystallines, liquid crystals, micelles, reverse micelles, emulsions, and microemulsion. The excipients serving as sustained release agents that are also incorporated into the porous excipients to form the MSR units include wax, sugar esters, semi-solid and solid alcohols (e.g. stearyl alcohol andcetyl alcohols), glyceryl behenate, mono-glycerol, di-glycerols, mon-glyceride, di-glyceride, triglyceride, shellac, any hydrophilic or hydrophobic materials with melting points lower than 120 degree Celsius, and or excipients that dissolved, at ambient or elevated temperature up to 100 degree Celsius, in ethanol, methanol, ether, methylene chloride, acetone, ethyl acetate, tetrahydrofuron (THF), water, di-methyl formamide, urea, hexane, pentane, butane, heptane, octane, xylene, toluene, pyridine, polyethylene glycols, oleic acid, dimethylacetamide, polysorbates, sorbitan monolaurate, sorbitan monopalmitate, sorbitan monostearate, sorbitan monooleate, or any combination thereof.

Once the MSR or MCR units are made, they can be used to construct the second part by mixing with proper ODT excipients such as superdisintegrants, defined as excipients that upon exposure to water will rapidly absorb water and swell, together with binders and then compressing them into orally disintegrating tablets (see FIG. 1B and the morphology of this part).

The ODT excipients include but not limited to mannitol, sucrose, sorbitol, xylitol, flutose, polyethylene glycol (PEG), hydroxypropylmethocellulose (HPMC), hydroxypropylcellulose (HPC), and other water swelling excipients. The binders can be polymeric, such as polylvinylpirrolidone (PVP), methylcellulose, starches, lactose, wax, emulsifying wax, Sorbitan monolaurate, Sorbitan monopalmitate, sorbitan monostearate, sorbitan tristearate, sorbitan monooleate, polyoxyethylene sorbitan monolaurate, polyoxyethylene, sorbitan monopalmitate, polyoxyethylene sorbitan monostearate, polyoxyethylene sorbitan monooleate, and other non-ionic polymeric surfactants, di-calcium phosphate, guar gums, acacia, and gelatins. The superdisintegrant candidates for rapidly swelling to take in water for dissolving the binders and bulking agents include but not limited to polyvinuylpirrolidone, croscarmellose sodium, Indion 414 (a weak acid cation exchange resin), etc.

### Example 1

Congo Red die was used as the model drug. The formulation compositions are given in Tablet 1.

TABLE 1
Formulation compositions of Congo Red ODT-SR tablet
using technology described in this invention.

| Ingredient | Weight(mg) |
|---|---|
| Congo Red - active ingredient | 3 |
| glyceryl behenate | 65 |
| Silica (Aerosil 300) | 100 |
| Mannitol | 250 |
| croscarmellose sodium | 9 |

The process included weighing 30 mg of Congo Red and dissolving in 1 gm of deionized water. The water-Congo Red solution was heated to assist Congo Red dissolution, if necessary. Once the Congo Red was dissolved, silica was added into the Congo Red solution, and mixed well. This was followed by drying the mixture in an oven at 90 degree Celsius until constant weight is obtained. Glyceryl behenate was added and the temperature was maintained at 85 degree Celsius under constant agitation for approximately 1 hour, to make sure there were no agglomerates and that the power remained free flowing. Finally, the powder was cooled to ambient temperature, and superdisintegrant and mannitol were added and the powder was pressed into tablets. Each tablet weighs approximately 400-450 mg. The tablets thus made disintegrated within 10 seconds when tested by using the USP <701> method, which is incorporated by reference.

The apparatus used for testing disintegration included a basket-rack assembly, a 1000-mL, low form beaker for the immersion fluid that was kept at 37 degree Celsius. The basket was raised and lowered in the immersion fluid at a constant frequency rate between 30 cycles per minute through a distance of 5.5 cm. The volume of the fluid in the vessel was such that at the highest point of the upward stroke the wire mesh remained at about 2.7 cm below the surface of the fluid and descends to about 3.0 cm from the bottom of the vessel on the downward stroke.

Congo Red release rate was tested using an orbital shaker in an enclosed environment maintained at 37 degree Celsius. A tablet thus made was put in a basket recommended in a USP apparatus I, then hanged in the middle of a glass jar containing 400 ml of water at 37 degree Celsius and constantly shook at 100 rpm. This shaking water jar simulated a dissolution study mimicking a standard USP apparatus I dissolution study. FIG. 2 show the Congo Red release rate. The release rate was measured by measuring the color of the dissolution media and compared with a set of standard Congo Red solution from 0.00075 mg/ml to 0.0075 mg/ml. FIG. 2 shows the release of Congo Red sustained for more than 6 hours and the amount released at 24 hours was about 97%.

### Example 2

3,4-diaminopyridine (DAP) was used as an example for ODT-SR technology invention here. There were two different 3,4-diaminopyridine (DAP) formulations (Formulation 1 and Formulation 2) produced using the ODT-SR technology. The percent composition of the API and excipients in formulation 1 are listed below in Table 2. Formulation 1 was prepared as follows. The active ingredient (DAP) was mixed with Eudragit 5100, and Eudragit L100 in proper amount of acetone to dissolve both API and Eudragits, followed by adding Aeroperl 300. After evaporating solvent, added the remaining excipients and mixed well for tablet press. Formulation 2 (the percent composition of the API and excipients in this formulation is given in Table 3) was prepared by heating DAP, Aeroperl 300, Compritol and Beewax to 80° C. for 1 hour, followed by adding/mixing the remaining excipients for tablet press.

The dissolution was performed using an orbital shaker. Each tablet is situated in a basket resembling the USP appratus-1 basket submersed in a glass jar containing 400 ml DI water with temperature kept at 37° C. Time points were taken at 0.5, 1, 2, 4, 6, and 8 hours. Table 4 and FIG. 3 show the dissolution results for these two formulations.

TABLE 2
DAP Formulation 1 compositions
DAP I (Formulation 1) - composition

| | | |
|---|---|---|
| DAP | 3,4-diaminopyridine | 5.58% |
| Eudragit S100 | methyl methacrylate copolymer | 5.60% |
| | (1:2) | |
| Eudragit L100 | methyl methacrylate copolymer | 5.74% |
| | (1:1) | |
| Aeroperl 300 | colloidal silicon dioxide | 25.24% |
| Pearlitol 200 SD | mannitol | 50.25% |
| Povidone XL-10 | polyvinylpyrrolidone | 4.55% |
| Ac-di-Sol | sodium croscarmellose | 2.12% |
| Magnesium Stearate | magnesium stearate | 0.91% |

TABLE 3
DAP Formulation 2 compositions
DAP II (Formulation 2) - composition

| | | |
|---|---|---|
| DAP | 3,4-diaminopyridine | 5.76% |
| Compritol | glyceryl behenate | 6.81% |
| Beewax | carnauba wax | 6.83% |
| Aeroperl 300 | colloidal silicon dioxide | 22.70% |
| Pearlitol 200 SD | mannitol | 49.70% |
| XL-10 | polyvinylpyrrolidone | 4.54% |
| Ac-di-Sol | sodium croscarmellose | 2.48% |
| MgS | magnesium stearate | 1.18% |

TABLE 4
Formulation 1 and 2 dissolution results

| | Formulation 1 | | Formulation 2 | |
|---|---|---|---|---|
| | Average | Standared | Average | Standared |
| Time (hr) | Absrobance | deviation | Absrobance | deviation |
| 0.5 | 39.60% | 8.69% | 27.36% | 8.54% |
| 1 | 61.00% | 4.08% | 54.27% | 6.10% |
| 2 | 81.74% | 9.49% | 85.38% | 3.68% |
| 4 | 94.95% | 5.75% | 91.95% | 1.65% |
| 6 | 99.61% | 0.55% | 96.12% | 0.33% |
| 8 | 98.81% | 1.68% | 100.00% | 0.00% |

### Example 3

Levodopa/Carbidopa combination drug with weight ratio of 4:1 was used as the API for producing ODT-SR Formulations. There were two types of formulations produced using the invention technology. The first type is ODT-SR (sustained release) and the second type is ODT-CR (controlled release). One formulation was made to test the ODT-SR and four formulations were made to test the ODT-CR (orally disintegrating controlled release), VRL0006-8i, VRL0006-8ii, VRL0006-8iii, and VRL0006-10. The percent compositions of the ODT-SR formulations are listed in Table 5. The ODT-SR formulation was prepared by heating Levodopa, Carbidopa, compritol in a scintillation vial to 80° C. for 1 hours, followed by adding the rest of excipients for tablet press.

TABLE 5
Composition of Levodopa/Carbidopa (4:1) ODR-SR formulation

| ODT-SR | Composition |
|---|---|
| Levodopa | 3.00% |
| Carbidopa | 0.76% |
| Aeroperl 300 | 22.08% |
| Compritol | 8.51% |
| Pearlitol 200 SD | 57.95% |
| Povidone XL-10 | 4.30% |
| Ac-Di-Sol | 2.13% |
| Magnesium Stearate | 1.28% |

The dissolution was performed using an orbital shaker. Each tablet is situated in a basket resembling the USP appratus-1 basket submersed in a glass jar containing 400 ml of 0.1% ascorbic acid in DI water (w/v). The dissolution was conducted at 37° C. Time points were taken at 0.5, 1, 2, 4, 6, and 7.67 hours. Table 6 and FIG. 4 show the dissolution results.

TABLE 6
Levodopa/Carbidopa ODT-SR dissolution result

| | Drug release | Standard deviation |
|---|---|---|
| Time (hr) | (%) | (%) |
| 0.5 | 21.72% | 7.37% |
| 1 | 44.92% | 13.10% |
| 2 | 77.82% | 10.29% |
| 4 | 94.41% | 2.88% |
| 6 | 93.68% | 4.73% |
| 7.67 | 96.13% | 3.90% |

The ODT-CR of Levodopa/Carbidopa controlled released formulations were prepared by mixing Levodopa and Carbidopa with Eudragit L100:S100 (1:4) in proper amount of acetone to dissolve both API and Eudragits, followed by adding Aeroperl 300. After evaporating solvent, added the remaining excipients and mixed well for tablet press. The compositions for the four ODT-CR formulations (VRL0006-8i, VRL0006-8ii, VRL0006-8iii, and VRL0006-10) are given in Table 7A and 7B.

TABLE 6A
Compositions of Levodopa/Carbidopa (weight
ratio = 4:1) ODR-CR formulations

| ODT-CR | VRL0006-8i | VRL0006-8ii | VRL0006-8iii |
|---|---|---|---|
| Levodopa | 0.53% | 0.62% | 0.63% |
| Carbidopa | 0.13% | 0.15% | 0.16% |
| Aeroperl 300 | 31.59% | 36.63% | 37.39% |
| Eudragit L100:S100 (1:4) | 20.22% | 11.75% | 6.89% |
| Pearlitol 200 SD | 39.38% | 43.07% | 47.11% |
| Povidone XL-10 | 4.75% | 4.53% | 4.63% |
| Ac-Di-Sol | 2.27% | 2.12% | 2.11% |
| Magnesium Stearate | 1.12% | 1.12% | 1.08% |

TABLE 6B
Composition of the fourth Levodopa/Carbidopa
(4:1) ODR-CR formulation

| ODT-CR | VRL0006-10 |
|---|---|
| Levodopa | 3.00% |
| Carbidopa | 0.76% |
| Aeroperl 300 | 22.08% |
| Carnauba Wax | 8.51% |
| Pearlitol 200 SD | 57.95% |
| XL-10 | 4.30% |
| Ac-Di-Sol | 2.13% |
| MgS | 1.28% |

The ODT-CR tablet dissolution was performed using an orbital shaker. Each tablet is situated in a basket resembling the USP appratus-1 basket submersed in a glass jar containing 400 ml of 0.1% ascorbic acid in DI water (w/v). The dissolution was conducted at 37° C. The time points taken were 0.5, 1, 2, and 4 hours. At the 1 hour time point 0.2M NaOH was added to the dissolution media until the dissolution media had a pH of about 7. Table 7 and FIG. 5 show the results.

TABLE 7
Levodopa/Carbidopa ODT-CR dissolution results

| | VRL0006-8i | | VRL0006-8ii | | VRL0006-8iii | | VRL0006-10 | |
|---|---|---|---|---|---|---|---|---|
| Time (hours) | Abs. 296 | Normalized | Abs. 296 | Normalized | Abs. 296 | Normalized | Abs. 296 | Normalized |
| 0.5 | −0.0247 | −0.0177315 | 0.0537 | 0.0375472 | 0.04416 | 0.030396476 | 0.02072 | 0.01481587 |
| 1 | −0.02168 | −0.0155635 | 0.04898 | 0.03424696 | 0.05136 | 0.035352423 | 0.04279 | 0.03059707 |
| 2 | 1.393 | 1 | 1.4302 | 1 | 1.4528 | 1 | 1.3985 | 1 |
| 4 | 1.3233 | 0.94996411 | 1.4063 | 0.98328905 | 1.4426 | 0.992979075 | 1.3418 | 0.95945656 |

The tablet form was described in the examples but the orally disintegrating form is not limited to the tablet form. The orally disintegrating form may be made into pellets, granules, or beads, among others. For example, the compression process may be altered to produce pellets using small punches, granules using roller compactors followed by sieving through screens or a granulator, and beads using extruders followed by a spheronization process.

What is claimed is:

1. An orally disintegrating form comprising:
a first part containing an active ingredient; first excipients for sustained release;
and a pre-formed porous structure, wherein the active ingredient and the first excipients are incorporated into the pre-formed porous structures to form micron size sustained release units; and
a second part including an orally disintegrating excipient with a binder, wherein the first part and the second part are compressed together to form final dosage forms.

2. The form according to claim 1, wherein
the first excipient comprises wax, sugar esters, semi-solid and solid alcohols (e.g. stearyl alcohol andcetyl alcohols), glyceryl behenate, mono-glycerol, di-glycerols, mon-glyceride, di-glyceride, triglyceride, shellac, any hydrophilic or hydrophobic materials with melting points lower than 120 degree Celsius, and/or excipients that dissolved, at ambient or elevated temperature up to 100 degree Celsius, in ethanol, methanol, ether, methylene chloride, acetone, ethyl acetate, tetrahydrofuron (THF), water, di-methyl formamide, urea, hexane, pentane, butane, heptane, octane, xylene, toluene, pyridine, polyethylene glycols, oleic acid, dimethylacetamide, polysorbates, sorbitan monolaurate, sorbitan monopalmitate, sorbitan monostearate, sorbitan monooleate, or any combination thereof.

3. The form according to claim 1, wherein the pre-formed porous structure comprises silica, polymers, mannitols, sorbitols, xylitols, fructose, lactose, starch, celluloses, guar gum, gelatins, Bucky balls, liposome, crystalline, polycrystallines, liquid crystals, micelles, reverse micelles, emulsions, and microemulsion.

4. The form according to claim 1, wherein the orally disintegrating excipient comprises mannitol, sucrose, sorbitol, xylitol, flutose, polyethylene glycol (PEG), hydroxypropylmethocellulose (HPMC), hydroxypropylcellulose (HPC), and other water swelling excipients or any combination thereof; and wherein the binder comprises polylvinylpirrolidone (PVP), methylcellulose, starches, lactose, wax, emulsifying wax, Sorbitan monolaurate, Sorbitan monopalmitate, sorbitan monostearate, sorbitan tristearate, sorbitan monooleate, polyoxyethylene sorbitan monolaurate, polyoxyethylene, sorbitan monopalmitate, polyoxyethylene sorbitan monostearate, polyoxyethylene sorbitan monooleate, and other non-ionic polymeric surfactants, di-calcium phosphate, guar gums, acacia, and gelatins, or any combination thereof.

5. The form according to claim 1, wherein the orally disintegrating excipient is a superdisintegrant which comprises polyvinuylpirrolidone, croscarmellose sodium, Indion 414, or a weak acid cation exchange resin.

6. The form according to claim 3, wherein
the size of the pre-formed porous structure ranges from about 5 microns to about 500 microns.

7. The form according to claim 1, wherein the form is a tablet, bead, pellet, or granule.

8. A method of making an orally disintegrating form comprising:
providing a first part containing an active ingredient; first excipients for sustained release;
and a pre-formed porous structure, wherein the active ingredient and the first excipients are incorporated into the pre-formed porous structures to form micron size sustained release units; and
providing a second part including orally disintegrating excipients with binders, wherein the first part and the second part are compressed together to form tablets, granules, beads, pellets, or any solids of no less than about 500 micrometers in diameter.

9. The method according to claim 8, wherein the first excipient comprises wax, sugar esters, semi-solid and solid alcohols (e.g. stearyl alcohol andcetyl alcohols), glyceryl behenate, mono-glycerol, di-glycerols, mono-glyceride, di-glyceride, triglyceride, shellac, any hydrophilic or hydrophobic materials with melting points lower than 120 degree Celsius, and/or excipients that dissolved, at ambient or elevated temperature up to 100 degree Celsius, in ethanol, methanol, ether, methylene chloride, acetone, ethyl acetate, tetrahydrofuron (THF), water, di-methyl formamide, urea, hexane, pentane, butane, heptane, octane, xylene, toluene, pyridine, polyethylene glycols, oleic acid, dimethylacetamide, polysorbates, sorbitan monolaurate, sorbitan monopalmitate, sorbitan monostearate, sorbitan monooleate, or any combination thereof.

10. The method according to claim 8, wherein
the pre-formed porous structure comprises silica, polymers, mannitols, sorbitols, xylitols, fructose, lactose, starch, celluloses, guar gum, gelatins, Bucky balls, liposome, crystalline, polycrystallines, liquid crystals, micelles, reverse micelles, emulsions, and microemulsion.

11. The method according to claim 8, wherein the orally disintegrating excipient comprises mannitol, sucrose, sorbitol, xylitol, flutose, polyethylene glycol (PEG), hydroxypropylmethocellulose (HPMC), hydroxypropylcellulose (HPC), and other water swelling excipients or any combination thereof; and wherein the binder comprises polylvinylpirrolidone (PVP), methylcellulose, starches, lactose, wax, emulsifying wax, Sorbitan monolaurate, Sorbitan monopalmitate, sorbitan monostearate, sorbitan tristearate, sorbitan monooleate, polyoxyethylene sorbitan monolaurate, polyoxyethylene, sorbitan monopalmitate, polyoxyethylene sorbitan monostearate, polyoxyethylene sorbitan monooleate, and other non-ionic polymeric surfactants, di-calcium phosphate, guar gums, acacia, and gelatins, or any combination thereof.

12. The method according to claim 8, wherein the orally disintegrating excipient is a superdisintegrant which comprises polyvinuylpirrolidone, croscarmellose sodium, Indion 414, or a weak acid cation exchange resin.

13. The method according to claim 8, wherein
the size of the pre-formed porous structure ranges from about 5 microns to about 500 microns.

14. The method according to claim 8, wherein the form is a tablet, bead, pellet, or granule.
